(19) United States
(12) Patent Application Publication
(10) Pub. No.: US 2026/0131086 A1
(43) Pub. Date: May 14, 2026

(54) A SUB-ASSEMBLY OF A MEDICAMENT DELIVERY DEVICE

(71) Applicant: SHL Medical AG, Zug (CH)

(72) Inventors: Tai-Fu Wang, Taoyuan City (TW); Chun-Hsien Huang, Taoyuan City (TW); Ming-Ting Yin, Taoyuan City (TW); Hamza Azam, Taoyuan City (TW)

(21) Appl. No.: 19/119,900

(22) PCT Filed: Oct. 5, 2023
(86) PCT No.: PCT/EP2023/077554

§ 371 Date: Apr. 10, 2025

Related U.S. Application Data
(60) Provisional application No. 63/417,717, filed on Oct. 20, 2022.
(60) Provisional application No. 63/430,743, filed on Dec. 7, 2022.

(30) Foreign Application Priority Data
Nov. 21, 2022 (EP) 22208474.1

(51) Int. Cl. A61M 5/32; A61M 5/31

(52) U.S. Cl. CPC A61M 5/3202; A61M 5/3137; A61M 2205/0205; A61M 2205/8212

(57) ABSTRACT

The present disclosure provides a sub-assembly of a medicament delivery device. The sub-assembly includes a housing having a housing body extending along a longitudinal axis between a proximal end and a distal end. The housing body is configured to accommodate a medicament container. The sub-assembly further includes a cap assembly comprising a cap body. The cap body is releasably attached to the proximal end of the housing body. The cap body is configured to cover a medicament delivery member when the cap body is attached to the proximal end of the housing body. The cap assembly comprises a set of electronics comprising a battery, a circuit and a switch comprising a distally directed surface. The set of electronics is attached to the cap body. The switch is operable between an active state where the circuit is connected to the battery and an inactive state where the circuit is disconnected from the battery. The housing comprises a proximally directed surface engaged with the distally directed surface of the switch when the cap body is attached to the housing body such that the switch is in the inactive state when the proximally directed surface is engaged with the distally directed surface of the switch.

### TECHNICAL FIELD

The present disclosure generally relates to a sub-assembly of a medicament delivery device, and particularly to a sub-assembly of a medicament delivery device comprising a switch.

### BACKGROUND

Medicament delivery devices such as auto-injectors, inhalers, or on-body devices are generally known for the self-administration of a medicament by patients without formal medical training. For example, patients suffering from diabetes or people who are undergoing an artificial fertilization procedure may require repeated injections of insulin or hormone. Other patients may require regular injections of other types of medicaments, such as a growth hormone.

As those medicament delivery devices are designed for patients without formal medical training and operation of those medicament delivery devices might be taking place in a patient's own house, which is usually not in a place of professional health/medical care, e.g. hospital, clinic or health centres, there is a demand for automatically recording every single delivery operation that has been taken by the user. The record can help the user to track his/her medicament intake or as the basis of an alarm as the next operation reminder; the record can also help a medical doctor or a health care provider to track the compliance of the user regarding the therapeutic regimen. Furthermore, to ensure users'safety in accessing/using a medicament delivery device, there is also a demand for stopping users from accessing or using a medicament delivery device which has been used.

Electronic medicament delivery devices have been developed for allowing patients themselves to safely administer medicament, without the need for help from health professionals, and for allowing transmission of data to the health professionals. Data is generally transmitted by an electronic component that is powered by a battery integrated within the device or through a wired connection by an external power source.

Nowadays, the material cost of electronics that can provide the above-mentioned functions is reducing. However, the cost of manufacture is increasing, by either the increasing cost of manpower or the increasing cost of making a more complicated automatic assembling machine, so there is still room for simplifying the assembling process for medicament delivery devices.

### SUMMARY

The invention is defined by the appended claims, to which reference should now be made.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

There is hence provided a sub-assembly of a medicament delivery device. The medicament delivery device comprises a medicament container containing a medicament and a medicament delivery member operably connected to the medicament container for delivering the contained medicament. The sub-assembly comprises: a housing having a housing body extending along a longitudinal axis between a proximal end and a distal end; wherein the housing body is configured to accommodate the medicament container; and a cap assembly comprising a cap body; wherein the cap body is releasably attached to the proximal end of the housing body. The cap body is configured to cover the medicament delivery member when the cap body is attached to the proximal end of the housing body. The cap assembly comprises a set of electronics comprising a battery, a circuit and a switch comprising a distally directed surface. The set of electronics is attached to the cap body. The switch is operable between an active state where the circuit is connected to the battery and an inactive state where the circuit is disconnected from the battery. The housing comprises a proximally directed surface engaged with the distally directed surface of the switch when the cap body is attached to the housing body such that the switch is in the inactive state when the proximally directed surface is engaged with the distally directed surface of the switch and is in the active state when the proximally directed surface is disengaged from the distally directed surface of the switch.

Preferably, according to another embodiment, the set of electronics comprises a biasing member configured to bias the switch from the inactive state to the active state.

Preferably, according to another embodiment, the proximally directed surface of the housing is configured to press the switch against the biasing force from the biasing member when the proximally directed surface of the housing is engaged with the distally directed surface of the switch.

Preferably, according to another embodiment, the switch is pivotal between the inactive state and the active state.

Preferably, according to another embodiment, the switch is pivotal on a plane parallel to the longitudinal axis.

Preferably, according to another embodiment, the housing comprises a protrusion extending in a direction transverse to the longitudinal axis. The proximally directed surface is defined by the protrusion.

Preferably, according to another embodiment, the protrusion is attached to the housing body.

Preferably, according to another embodiment, the protrusion is immovably attached to the housing body such that the protrusion is rotationally and axially immovable relative to the housing body.

Preferably, according to another embodiment, the set of electronics is embedded in the cap body.

Preferably, according to another embodiment, the cap body comprises an outer surface facing away from the longitudinal axis in the direction transverse to the longitudinal axis.

Preferably, according to another embodiment, the set of electronics is attached to the outer surface of the cap body.

Preferably, according to another embodiment, the cap assembly comprises an electronic housing.

Preferably, according to another embodiment, the set of electronics is received within the electronic housing.

Preferably, according to another embodiment, the electronic housing comprises a distally directed opening at least partially aligned with the distally directed surface of the switch.

Preferably, according to another embodiment, the electronic housing is attached to the cap body.

Preferably, according to another embodiment, the electronic housing is immovably attached to the cap body such that the electronic housing is rotationally and axially immovable relative to the cap body.

Preferably, according to another embodiment, the protrusion is at least partially positioned within the distally directed opening when the cap body is attached to the housing body.

Preferably, according to another embodiment, the set of electronics comprises a communication unit connected to the circuit.

Preferably, according to another embodiment, the communication unit is configured to wirelessly send out and/or receive a signal when the circuit is connected to the battery.

Preferably, according to another embodiment, the sub-assembly comprises a delivery member guard telescopically arranged relative to the proximal end of the housing. The delivery member guard is configured to surround the medicament delivery member once the cap body is removed from the housing.

Preferably, according to another embodiment, the sub-assembly is used in a medicament delivery device. The medicament delivery device comprises a medicament container containing medicament, and a medicament delivery member operably connected to the medicament container for delivering the contained medicament.

Preferably, according to another embodiment, the medicament delivery member of the medicament delivery device is a needle or a spray nozzle.

Preferably, according to another embodiment, the medicament container of the medicament delivery device is a syringe, a cartridge or a collapsible bag.

Preferably, according to another embodiment, the medicament container of the medicament delivery device is made of glass material or plastic material.

Preferably, according to another embodiment, the medicament delivery device is an injection device, an inhalation device, or a medical sprayer.

Preferably, according to another embodiment, the medicament delivery device is an auto-injector.

Preferably, according to another embodiment, the medicament delivery device is a hand-held, pen-type auto-injector.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of drugs that could be included in the medicament delivery devices described herein include, but are not limited to, small molecules, hormones, cytokines, blood products, antibodies, antibody-drug conjugates, bispecific antibodies, proteins, fusion proteins, peptibodies, polypeptides, pegylated proteins, protein fragments, protein analogues, protein variants, protein precursors, chimeric antigen receptor T cell therapies, cell or gene therapies, oncolytic viruses, or immunotherapies and/or protein derivatives. Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)), ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab. Pharmaceutical formulations including, but not limited to, any drug described herein are also contemplated for use in the medicament delivery devices described herein, for example, pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

Exemplary drugs that could be included in the medicament delivery devices described herein include, but are not limited to, an immuno-oncology or bio-oncology medications such as immune checkpoints, cytokines, chemokines, clusters of differentiation, interleukins, integrins, growth factors, enzymes, signaling proteins, pro-apoptotic proteins, anti-apoptotic proteins, T-cell receptors, B-cell receptors, or costimulatory proteins.

Exemplary drugs that could be included in the medicament delivery devices described herein include, but are not limited to, those exhibiting a proposed mechanism of action, such as HER-2 receptor modulators, interleukin modulators, interferon modulators, CD38 modulators, CD22 modulators, CCR4 modulators, VEGF modulators, EGFR modulators, CD79b modulators, Trop-2 modulators, CD52 modulators, BCMA modulators, PDGFRA modulators, SLAMF7 modulators, PD-1/PD-L1 inhibitors/modulators, B-lymphocyte antigen CD19 inhibitors, B-lymphocyte antigen CD20 modulators, CD3 modulators, CTLA-4 inhibitors, TIM-3 modulators, VISTA modulators, INDO inhibitors, LAG3 (CD223) antagonists, CD276 antigen modulators, CD47 antagonists, CD30 modulators, CD73 modulators, CD66 modulators, CDw137 agonists, CD158 modulators, CD27 modulators, CD58 modulators, CD80 modulators, CD33 modulators, APRIL receptor modulators, HLA antigen modulators, EGFR modulators, B-lymphocyte cell adhesion molecule modulators, CDw123 modulators, Erbb2 tyrosine kinase receptor modulators, mesothelin modulators, HAVCR2 antagonists, NY-ESO-1 OX40 receptor agonist modulators, adenosine A2 receptors, ICOS modulators, CD40 modulators, TIL therapies, or TCR therapies.

Exemplary drugs that could be included in the medicament delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOPAD, 7+3, 5+2 , 7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VelP, OFF, GTX, CAV, AD, MAID, AIM, VAC-IE, ADOC, or PE.

Exemplary drugs that could be included in the medicament delivery devices described herein include, but are not limited to, those used for chemotherapy, such as an alkylating agent, plant alkaloid, antitumor antibiotic, antimetabolite, or topoisomerase inhibitor, enzyme, retinoid, or corticosteroid. Exemplary chemotherapy drugs include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

Furthermore, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### BRIEF DESCRIPTION OF THE DRAWINGS

Embodiments of the inventive concept will now be described, by way of example only, with reference to the accompanying drawings, in which:

FIG. 1 schematically shows a perspective view of a medicament delivery device with a sub-assembly of the invention;

FIG. 2 schematically shows a cross-section view of a proximal portion of FIG. 1;

FIG. 3 schematically shows a perspective view of a switch of a set of electronics of the sub-assembly of the invention and a protrusion of a housing of the sub-assembly of the invention;

FIG. 4 schematically shows a perspective view of the switch of FIG. 3;

FIG. 5 schematically shows a perspective view of a housing connector of the sub-assembly of the invention;

FIGS. 6-7 schematically shows a perspective view of an electronic housing of the sub-assembly of the invention;

FIG. 8 schematically shows a perspective view of the sub-assembly of the invention;

FIG. 9 schematically shows a perspective view of the sub-assembly of the invention when the cap body is removed from the housing body;

FIG. 10 schematically shows a perspective view of a way of recycling the medicament delivery device of FIG. 1.

FIGS. 11-13 schematically show another example of the sub-assembly of the invention with a secondary safety feature.

### DETAILED DESCRIPTION

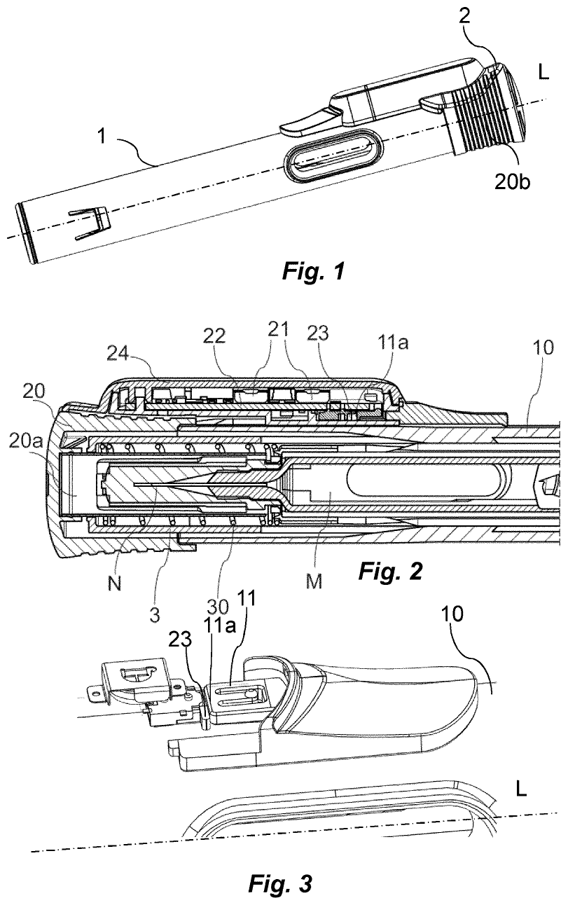

FIGS. 1-10 illustrate a medicament delivery device. The medicament delivery device comprises a medicament container M containing a medicament and a medicament delivery member N operably connected to the medicament container M for delivering the contained medicament. In one example, the medicament container M is a syringe, and the medicament delivery member N is a needle. In this example, the needle is integral at a proximal end of the syringe. Alternatively, the medicament container can be a cartridge, and the medicament delivery member N is a needle that is preassembled to the medicament delivery device and is configured to fluidly communicated with the cartridge during use. Alternatively, the medicament container M is a collapsible bag. For example, the medicament delivery device comprises a container carrier. In one example, the medicament container is made of plastic.

The sub-assembly comprises a housing 1 and a cap assembly 2. The housing 1 has a housing body 10 extending along a longitudinal axis L between a proximal end and a distal end, as shown in FIG. 1. The housing body 10 is configured to accommodate the medicament container M, as shown in FIG. 2. In a preferred example, the housing body 10 is shaped to match the shape of the medicament container M. For example, most of the medicament containers have a cylindrical body. Therefore, the housing body 10 is shaped to be cylindrical. Additionally, the housing body might comprise an anti-rolling feature that is configured to prevent the housing body from rolling on a flat surface. In one example, the anti-rolling feature can be one or more protrusions extending from an outer surface of the housing body. Alternatively, the anti-rolling feature can be provided by the housing body being shaped with a portion that has a non-circular cross-section when observed along the longitudinal axis L. For example, the housing body might have a triangle shaped cross-section. The sub-assembly optionally comprises a delivery member guard 3. The delivery member guard 3 is telescopically arranged relative to the housing body, e.g., being based in the proximal direction relative to the housing by a delivery member guard spring 30. In a preferred example, the delivery member guard 3 is arranged within the housing body 10, as shown in FIG. 2. In a preferred example, the delivery member guard 3 is shaped to match the shape of the housing body. In a preferred example, the delivery member guard 3 is tubular and is arranged between an inner wall of the housing body and the medicament container in a direction transverse to the longitudinal axis L.

The cap assembly 2 comprises a cap body 20. The cap body 20 is releasably attached to the proximal end of the housing body 10. The cap body 20 is configured to cover the medicament delivery member N when the cap body 20 is attached to the proximal end of the housing body 10. As a result, the user can be prevented from getting injured by the medicament delivery member N, e.g., being stuck by the needle, also, the medicament delivery member N can be prevented from being contaminated by the environment. As the needle is commonly sealed by a needle shield, in one example where the medicament delivery device is a syringe with a needle, the cap body comprises a needle shield remover 20a configured to surround and grip the needle shield such that when the user removes the cap body 20 from the housing body 10, the needle shield is removed from the needle. In one example, the cap body 20 comprises a fastener configured to engage with a counter fastener of the housing body 10 so that the cap body is attached to the housing body 10.

Alternatively, the cap body is attached to the housing body via the needle shield remover 20a. As a contact surface between the needle shield and the needle generates friction, e.g., 3-4 Newton, the cap body can be attached to the housing body via the connection between the needle shield remover and the needle shield. Alternatively, in one example where the sub-assembly comprises the delivery member guard, the fastener of the cap body is configured to engage with a counter fastener of the delivery member guard.

In one example, the cap body 20 comprises a tubular interior as shown in FIG. 2. The cap body can be shaped to match the shape of the housing body so that the medicament delivery device comprising the sub-assembly can be compact.

Additionally, the cap body 20 comprises one or more gripping assisting features to facilitate the user to easily grip and remove the cap body 20 from the housing body 10. The gripping assisting feature can be one or more flanges, a ring-pull, or multiple annular or curved protrusions 20b extending circumferentially and transversally as shown in FIG. 1. Additionally, the gripping assisting feature can be made of anti-slip material.

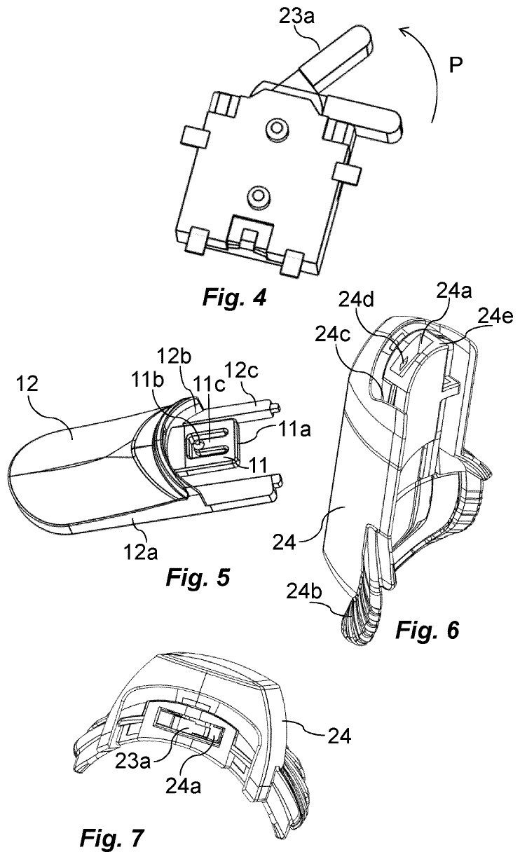

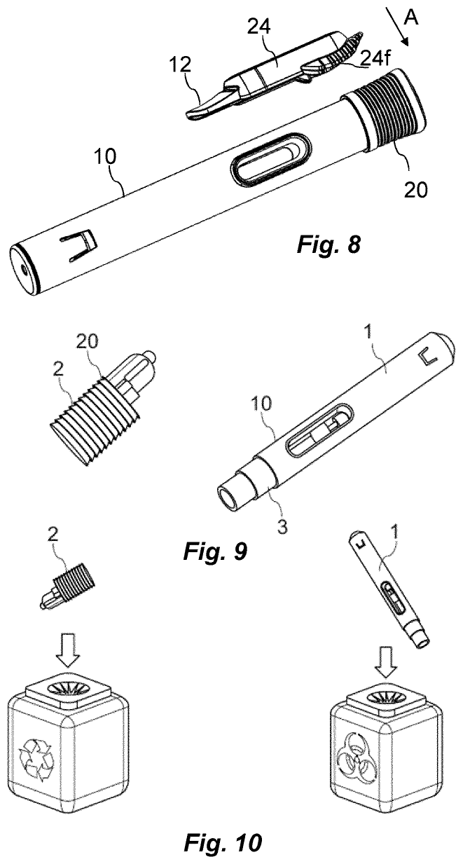

The cap assembly 2 comprises a set of electronics comprising a battery 21, a circuit 22 and a switch 23 comprising a distally directed surface 23a, as shown in FIGS. 2 and 4. The set of electronics is attached to the cap body 20. In one example, the set of electronics is embedded in the cap body. Alternatively, the set of electronics is attached to an outer surface of the cap body 20, as shown in FIG. 8. In this example, the cap body 20 comprises the outer surface facing away from the longitudinal axis L in the direction transverse to the longitudinal axis L.

In one example where the set of electronics is attached to the outer surface of the cap body 20, the cap assembly preferably comprises an electronic housing 24. The set of electronics is arranged within the electronic housing 24. In a preferred example, the electronic housing 24 comprises a distally directed opening 24a at least partially aligned with the distally directed surface 23a of the switch 23, as shown in FIG. 7. The electronic housing 24 is attached to the cap body 20. In one example where the cap body comprises the gripping assisting feature 20b, the electronic housing 24 can also be arranged with a gripping assisting feature 24b matched the gripping assisting feature 20b of the cap body 20. For example, when the gripping assisting feature 20b of the cap body 20 is multiple annular protrusions, the gripping assisting feature 24b of the electronic housing 24 might also be arranged with similar protrusions. In a preferred example, the electronic housing 24 is immovably attached to the cap body 20 such that the electronic housing is rotationally and axially immovable relative to the cap body 20.

The switch 23 is operable between an active state where the circuit 22 is connected to the battery 21 and an inactive state where the circuit 22 is disconnected from the battery 21. The housing 1 comprises a proximally directed surface 11a engaged with the distally directed surface 23a of the switch 23 when the cap body 20 is attached to the housing body 10 such that the switch 23 is in the inactive state when the proximally directed surface 11a is engaged with the distally directed surface 23a of the switch 23 and is in the active state when the proximally directed surface 11a is disengaged from the distally directed surface 23a of the switch 23. Therefore, the switch 23 will be inactive before the cap body 20 is removed from the housing body 10, thus battery consumption can be minimized.

In one example, the set of electronics comprises a biasing member configured to bias the switch from the inactive state to the active state. The biasing member can be a compression spring, a torsion spring, or a leaf spring, for example. The proximally directed surface 11a of the housing 1 is configured to press the switch 23 against the biasing force from the biasing member when the proximally directed surface 11a of the housing 1 is engaged with the distally directed surface 23a of the switch 23. In a preferred example, the switch 23 is pivotable between the inactive state and the active state, as shown in FIG. 4. The switch is moved from the inactive state to the active state via a pivotal movement P. The pivotal movement can be caused by a torsion spring or a leaf spring, for example. In a preferred example, the switch 23 is moved from the inactive state to the active state along a pivotal path; the pivotal path is parallel to a plane that is parallel to the longitudinal axis L. Alternatively, the switch is movable in a direction of the longitudinal axis L between the inactive state and the active state.

In one example, the housing 1 comprises a protrusion 11 extending in the direction transverse to the longitudinal axis L. The proximally directed surface 11a is defined by the protrusion 11, as shown in FIG. 3. In one example, the protrusion is integral to the housing body. In one example where the housing body comprises one or more protrusions as the anti-rolling features, the protrusion 11 that comprises the proximally directed surface 11a for engaging with the distally directed surface 23a of the switch 23 can be one of the anti-rolling protrusions.

Alternatively, the protrusion 11 is attached to the housing body 10, as shown in FIG. 8. In other words, in this example, the protrusion and the housing body are made as two independent components; the protrusion is configured to be attached to the housing body before use of the medicament delivery device. In one example, the protrusion 11 is immovably attached to the housing body 10 such that the protrusion 11 is rotationally and axially immovable relative to the housing body 10.

In one example where the protrusion 11 is immovably attached to the housing body 10 and the set of electronics is immovably attached to the cap body 20, the cap body 20 can be removed from the housing body 10 via a linear pulling movement in the proximal direction relative to the housing body 10. For example, the housing body comprises an elongated rib extending in the direction of the longitudinal axis L, and the cap body comprises a recess/cutout receiving the elongated rib of the housing body. Thus, the cap assembly is prevented from rotating relative to the housing body. Alternatively, the cap body can be removed from the housing body via a helical movement in the proximal direction relative to the housing body or via a rotation around the longitudinal axis relative to the housing body followed by a linear pulling movement in the proximal direction relative to the housing body. For example, the fastener of the cap body and the counter fastener of the housing body form a bayonet or a screw connection.

It should be noted that arranging the protrusion movable relative to the housing body and/or arranging the electronic housing movable relative to the cap body can be used to decide the exact point that the switch will be switched on and/or make the sub-assembly easy to be used. For example, when the cap body is configured to be removed from the housing body by rotating the cap body around the longitudinal axis relative to the housing body, the electronic housing can be axially immovable relative to and rotatably attached to the cap body such that when the cap body is rotated relative to the housing, the electronic housing is not rotated relative to the housing. Thus, the switch will be in the inactive state when the proximally directed surface of the housing is engaged with the distally directed surface of the switch; the switch with only move to the active state when the cap body is axially moved away from the housing body. Additionally, the electronic housing can be axially movably attached to the cap body. In this example, the cap body may move axially a certain distance, after which the electronic housing will be moved axially, e.g., by engaging with an edge of the cap body.

In one example where the protrusion 11 is attached to the housing 1 and the cap assembly comprises the electronic housing 24 attached to the cap body 20, the protrusion 11 is at least partially positioned within the distally directed opening 24a when the cap body 20 is attached to the housing body 10. In this example, the protrusion 11 together with the electronic housing 24 are configured to cover the switch 23 from the environment before the cap body 20 is removed from the housing body 10 such that on or more of environmental static electricity, dust, and dripping water cannot contaminate or impact on the switch 23 and thus damage the circuit. In a preferred example, the electronic housing 24 comprises only one opening which is the distally directed opening 24a, which is configured to be covered by the protrusion 11 of the housing 1 such that an ingress protection code IP22 protection can be provided to the set of electronics.

Furthermore, in another example, the sub-assembly comprises a housing connector 12, as shown in FIG. 5. The housing connector 12 comprises the protrusion 11. The housing connector 12 comprises a base 12a configured to be engaged with the outer surface of the housing body 10. In a preferred example, the base 12a is partially arranged within a receiving section 24c of the electronic housing 24. For example, the base 12a comprises a guide pin 12c configured to be positioned within the receiving section 24c of the electronic housing 24. The housing connector 12 comprises a contact wall 12b configured to be in contact with the counter contact wall 24e of the electronic housing 24. In one example, the contact wall 12b comprises a proximally directed surface configured to be in contact with a distally directed surface of the counter contact wall 24e. The protrusion 11 extends from the contact wall 12b in the proximal direction. In one example, the protrusion 11 comprises a fixture 11b and the electronic housing 24 comprises a counter fixture 24d. In one example, the protrusion 11 comprises a flexible arm 11c that is flexible in the direction transverse to the longitudinal axis L, as shown in FIG. 5. In this example, the fixture 11b and the counter fixture 24d form a detachable snap-fit connection.

In one example, the electronic housing 24 comprises a surface 24f configured to match the shape of the cap body 20. For example, when the cap body 20 has a curved outer surface 24f, the surface of the electronic housing is curved as shown in FIG. 7. In one example, the electronic housing 24 is configured to be adhesively attached to the cap body 20. In another example, the housing connector 12 is configured to be adhesively attached to the housing body 10.

In one example where the sub-assembly comprises the electronic housing 24 and the housing connector 12, the sub-assembly can be assembled with the following steps:
providing an assembly comprising the electronic housing 24 and the housing connector 12. The electronic housing 24 and the housing connector 12 are configured to being attached to one another via the connection between the fixture 11b of the housing connector 12 and the counter fixture 24d of the electronic housing 24;
aligning the surface 24f of the electronic housing 24 to an outer surface of the cap body 20 in the direction transverse to the longitudinal axis L;
moving the assembly transversally towards the cap body 20 and the housing body 10 (as shown with arrow A in FIG. 8) until the surface 24f of the electronic housing 24 is adhesively attached to the outer surface of the cap body 20 and the base 12a of the housing connector 12 is adhesively attached to the outer surface of the housing body 10.

The above-mentioned assembling steps can be carried out in a production line or by a care giver, e.g., a doctor, nurse, or pharmacist in a medical service facility, e.g., a hospital or a pharmacy.

FIG. 9 shows the cap body 20 removed from the housing body 10. In one example where the sub-assembly comprises the delivery member guard 3, the delivery member guard 3 is configured to surround and cover the delivery member when the cap body 20 is removed from the housing body 10. As the housing body 10 is configured to accommodate the medicament container and the delivery member, and the cap assembly comprises the set of electronics, when the medicament delivery device comprising the sub-assembly is used, as shown in FIG. 10, the housing 1 with the medicament container and the delivery member can be processed as medical waste, and the cap assembly with the set of electronics can be recycled easily. In one preferred example, the electronic housing 24 is configured to be attached to the cap body via glue or double-sided tape so that the electronic housing can be easily detached for recycling purposes.

In one example, the set of electronics comprises a communication unit connected to the circuit. The communication unit is configured to wirelessly send out, e.g. broadcast, and/or receive a signal when the circuit is connected to the battery.

The communication unit can be a short-range communication unit, such as RFID, NFC, infra-red, ZigBee, Bluetooth, and/or a long-range communication unit, such as 3G, 4G, CAT-M1, NB-IoT, LoRa, Sigfox, 5G, or GPRS.

In one example, the set of electronics comprises at least one of processor, e.g., an micro control unit (MCU), a memory, a clock, a communication unit, an indicator and or a sensor,

In one example, the memory can be a random access memory (RAM), a read-only memory (ROM), an erasable programmable read-only memory (EPROM), an electrically erasable programmable read-only memory (EEPROM) or a Flash memory, such as a compact Flash memory.

In one example, the indicator can be an acoustic indicator, such as a speaker or a buzzer. For example, the indicator can be a visual indicator, such as an e-ink display, an LCD display or a LED light emitter and/or the indicator can be a haptic indicator, such as a vibrator.

In one example, the sensor can be an orientation sensor, such as an accelerometer or a gyroscope, and/or the sensor can be an environmental condition sensor, such as a temperature sensor, vibration sensor or contact sensor.

In one example, the battery is a coin-sized battery, a thin film battery, and/or a fuel cell battery, e.g., an enzymatic paper-based fuel cell.

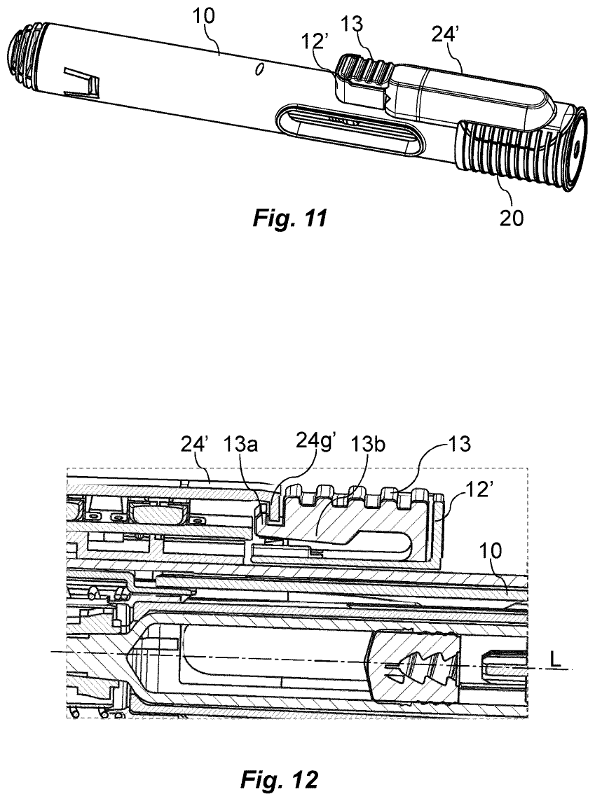

In another preferred example, the sub-assembly can be arranged with a secondary safety feature. In one example, as shown in FIG. 11, the base 12′comprises a button 13. The button 13 is configured to be pressed by the user before removing the cap body 20 from the housing body 10. Therefore, the unintentional cap removal can be prevented. In one example, as shown in FIG. 12, the button 13 comprises a flexible portion 13b being flexible in the direction of the longitudinal axis L and a hook 13a arranged at a proximal end of the flexible portion 13b. The hook 13a comprises a distally directed surface engaged with a proximally directed surface 24g′ of the electric housing 24′when the cap body 20 is attached to the housing body 10 and the button 13 is not pressed. Therefore, the electric housing 24′cannot be moved relative to the base 12′by being moved in the proximal direction relative to the base 12′. Once the user presses the button 13 towards the longitudinal axis L, the hook 13a of the button 13 disengaged with the proximally directed surface 24g′, the electric housing 24′can be moved relative to the base 12′in the proximal direction relative to the base 12′.

It should be noted that the button can be alternatively arranged at the electric housing and hooks the base.

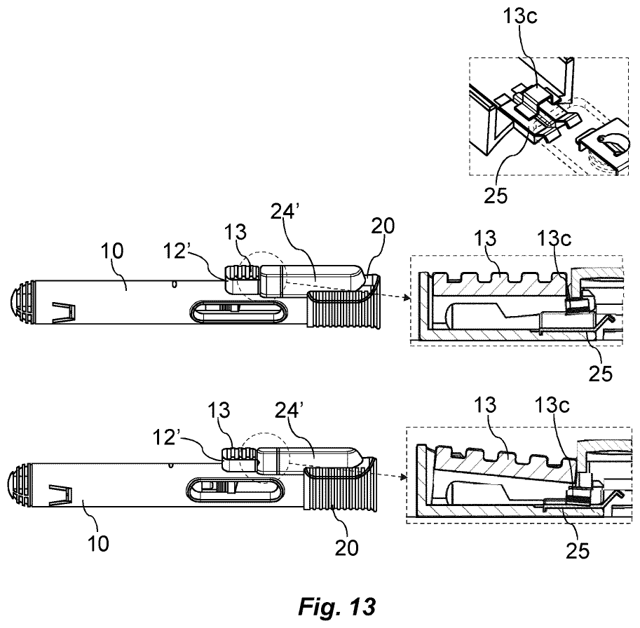

Furthermore, in one example as shown in FIG. 13, the button 13 comprises a conductive pad 13c. In this example, the set of electronics of electronics comprises a conductive join 25. In this example, when the button 13 is pressed, the conductive pad 13c is in contact with the conductive join 25. Therefore, a signal can be sensed by the circuit. This signal can be used as an initial trigger signal for the circuit, or only used for analyzing the user's behavior, e.g., whether the user uses the device correctly.

Other secondary safety feature can also be applied. For example, the base can comprise a conductive/inductive portion and the electric housing comprises a counter conductive/inductive portion. In this example, the conductive/inductive portion is away from the counter conductive/inductive portion initially. Once the cap body is moved relative to the housing body, the electric housing moves relative to the base. Thus, the conductive/inductive can move close or in contact with the counter conductive/inductive. Thus a signal is generated and can be detected by the circuit.

Furthermore, the housing, as mentioned in any example, may be provided with (i.e., molded in, molded with) a compound featuring persistently antimicrobial, antifungal, and/or antiviral properties. Alternatively, a compound featuring persistently antimicrobial, antifungal, and/or antiviral properties may be applied to the molded (i.e., finished) components through secondary processes (e.g., chemical vapor deposition), spraying, or dipping processes.

The inventive concept has mainly been described above with reference to a few examples. However, as is readily appreciated by a person skilled in the art, other embodiments than the ones disclosed above are equally possible within the scope of the inventive concept, as defined by the appended claims.

1-15. (canceled)

16. A sub-assembly of a medicament delivery device, wherein the medicament delivery device comprises a medicament container containing a medicament and a medicament delivery member operably connected to the medicament container for delivering the contained medicament, the sub-assembly comprising:
a housing having a housing body extending along a longitudinal axis between a proximal end and a distal end, wherein the housing body is configured to accommodate the medicament container; and
a cap assembly comprising a cap body, wherein the cap body is releasably attached to the proximal end of the housing body, wherein the cap body is configured to cover the medicament delivery member when the cap body is attached to the proximal end of the housing body,
wherein the cap assembly comprises a set of electronics comprising a battery, a circuit and a switch, wherein the switch comprises a distally directed surface, wherein the set of electronics is attached to the cap body, wherein the switch is operable between an active state where the circuit is connected to the battery and an inactive state where the circuit is disconnected from the battery, and wherein the housing comprises a proximally directed surface engaged with the distally directed surface of the switch when the cap body is attached to the housing body such that the switch is in the inactive state when the proximally directed surface is engaged with the distally directed surface of the switch and is in the active state when the proximally directed surface is disengaged from the distally directed surface of the switch.

17. The sub-assembly according to claim 16, wherein the set of electronics comprises a biasing member configured to bias the switch from the inactive state to the active state, and wherein the proximally directed surface of the housing is configured to press the switch against the biasing force from the biasing member when the proximally directed surface of the housing is engaged with the distally directed surface of the switch.

18. The sub-assembly according to claim 17, wherein the switch is pivotal between the inactive state and the active state.

19. The sub-assembly according to claim 18, wherein the switch is moved from the inactive state to the active state along a pivotal path, and wherein the pivotal path is parallel to a plane that is parallel to the longitudinal axis.

20. The sub-assembly according to claim 16, wherein the housing comprises a protrusion extending in a direction transverse to the longitudinal axis, and wherein the protrusion comprises the proximally directed surface.

21. The sub-assembly according to claim 20, wherein the protrusion is integral to the housing body.

22. The sub-assembly according to claim 20, wherein the protrusion is attached to the housing body.

23. The sub-assembly according to claim 22, wherein the protrusion is immovably attached to the housing body such that the protrusion is rotationally and axially immovable relative to the housing body.

24. The sub-assembly according to claim 16, wherein the set of electronics is embedded in the cap body.

25. The sub-assembly according to claim 16, wherein the cap body comprises an outer surface facing away from the longitudinal axis in a direction transverse to the longitudinal axis, and wherein the set of electronics is attached to the outer surface of the cap body.

26. The sub-assembly according to claim 25, wherein the cap assembly comprises an electronic housing, wherein the set of electronics is received within the electronic housing, wherein the electronic housing comprises a distally directed opening at least partially aligned with the distally directed surface of the switch, and wherein the electronic housing is attached to the cap body.

27. The sub-assembly according to claim 26, wherein the electronic housing is immovably attached to the cap body such that the electronic housing is rotationally and axially immovable relative to the cap body.

28. The sub-assembly according to claim 26, wherein the housing comprises a protrusion extending in a direction transverse to the longitudinal axis, wherein the protrusion comprises the proximally directed surface, and wherein the protrusion is at least partially positioned within the distally directed opening when the cap body is attached to the housing body.

29. The sub-assembly according to claim 16, wherein the set of electronics comprises a communication unit connected to the circuit, and wherein the communication unit is configured to wirelessly send out and/or receive a signal when the circuit is connected to the battery.

30. A medicament delivery device comprising a medicament container containing medicament, a medicament delivery member operably connected to the medicament container for delivering the contained medicament, and the sub-assembly according to claim 16.
